(19) United States
(12) Patent Application Publication
(10) Pub. No.: US 2018/0342324 A1
(43) Pub. Date: Nov. 29, 2018

(54) Systems and Methods for Creating Modular Data Processing Pipelines

(71) Applicant: pulseData Inc., New York, NY (US)

(72) Inventors: Theodore Cha, New York, NY (US); Chris Kipers, New York, NY (US); Edward Lee, Astoria, NY (US); Hai Po Sun, New York, NY (US)

(73) Assignee: pulseData Inc., New York, NY (US)

(21) Appl. No.: 15/992,104

(22) Filed: May 29, 2018

Related U.S. Application Data
(60) Provisional application No. 62/511,542, filed on May 26, 2017.
(60) Provisional application No. 62/545,617, filed on Aug. 15, 2017.

(51) Int. Cl. G16H 50/30; G16H 70/40; G16H 10/60; G16H 50/20; G16H 15/00; G06N 7/00; G06F 9/38

(52) U.S. Cl. CPC G16H 50/30; G16H 70/40; G16H 10/60; G06F 9/3867; G16H 15/00; G06N 7/005; G16H 50/20

(57) ABSTRACT

Systems, methods, and apparatuses are described herein that allow users to create and manage flexible, highly modular data processing pipelines. Such pipelines may be associated with any number of connected nodes connected via dependency injection to define the location and type of data that a pipeline uses as input or output and the operations to be performed by the pipeline. The pipelines may also be associated with context information, which specifies dataset-specific configurations and includes logic required to generate and execute the associated nodes. The context information may further include logic that allows for node substitution, caching of node output, data filtering, and/or dynamic node modification.

### CROSS-REFERENCE TO RELATED APPLICATIONS

The present application claims benefit of U.S. provisional patent application Ser. No. 62/511,542, titled “Systems and Methods for Creating Modular Data Processing Pipelines,” filed May 26, 2017 and U.S. provisional patent application Ser. No. 62/545,617, titled “Systems and Methods for Creating Modular Data Processing Pipelines,” filed Aug. 15, 2017. Each of the above applications is incorporated by reference herein in its entirety.

### BACKGROUND

This specification relates generally to data processing software. More specifically, this specification relates to applications, systems and methods for creating and managing flexible, maintainable and reusable data processing pipelines.

Predictive analytics is an emerging approach for disease treatment and prevention that uses data, statistical algorithms and machine learning techniques to identify the likelihood of future outcomes based on historical data. In healthcare applications, a primary goal of predictive analytics is to develop quantitative models for patients that can be used to determine current health status and to predict specific future events or developments, for example to assist healthcare professionals in treating or preventing disease or disability. In particular, for disease treatment and prevention, predictive analytics may take into account individual variability in genes, environment, health, and lifestyle.

The volume, variability and availability of electronic patient data has increased dramatically in recent years, including from sources such as electronic health records (“EHRs”), insurance claims, health facility and operations data (e.g., records relating to patient admission, discharge and/or transfer), lab results and genomics information. However, this data is not recorded in a state that provides a clear longitudinal or conceptual view of an individual patient's health. Accordingly, actionable prediction models may require a substantial number of multi-part calculations, assembling data from multiple heterogeneous data sources and assembling concepts out of a combination of individual data and metadata elements.

As an example, consider that a patient requires an estimated glomerular filtration rate (“eGFR”) score to, for example, measure the patient's level of kidney function and determine the patient's stage of kidney disease. In order to ascertain the eGFR score, calculations for determining the patient's average serum creatine level may be necessary. This problem requires a serial sequence of tasks, including matching a patient ID at multiple databases that hold historical serum creatine levels, invoking a Master Patient Index (“MPI”) to compress multiple IDs, assembling lab results into an eGFR score, determining the time of this score for the patient, and then calculating an average of serum creatine readings taken for the patient before this time. An arbitrary number of complicating layers can be added to this problem, for example calculating this same score for only patients in a certain demographic group. In solving this problem, there is a need to reapply complex calculations to new datasets in a transferable way, while allowing for dynamic modifications.

Example 1, below, shows pseudocode for an exemplary data processing pipeline that is similar to those that may be used in healthcare-related predictive analytics applications. As shown, the pipeline includes a number of functions (Functions 1-6) that invoke each other—Function 4 depends on Functions 1 and 2; Function 5 depends on Function 3; and Function 6 directly depends on Functions 4 and 5, and indirectly depends on Functions 1 and 2 (via Function 4) and Function 3 (via Function 5). Accordingly, Function 6 can be invoked without arguments to produce results that depend on each of Functions 1-5.

### EXAMPLE 1

```
function4( ){
  return function1( ) + function2( )
}
function5( ){
  return function3( )
}
function6( ){
  return function4( ) + function5( )
}
```

Although the exemplary pipeline of Example 1 may be used for simple functions and/or for small-numbers of functions; the exemplary pseudocode quickly becomes untenable as the number of parameters in a system increases. For example, assume that Functions 1-3 of Example 1 each require a file path parameter (e.g., “f1_file,” “f2_file,” and “f3_file,” respectively) to load data from an input file. In this case, there are two conventional approaches to accommodate the file path parameters.

The first conventional approach is to modify all of the functions to propagate the parameters to the correct functions, such as in Example 2, shown below. Unfortunately, the exemplary pseudocode of Example 2 creates brittle code that requires numerous modifications in all downstream functions whenever an upstream function introduces a new parameter. As such, this approach is not feasible for a large code base with multiple contributors.

### EXAMPLE 2

```
function4(f1_file, f2_file){
  return functionl(f1_file) + function2(f2_file)
}
function5(f3_file){
  return function3(f3_file)
}
function6(f1_file, f2_file, f3_file) {
  return function4(f1_file, f2_file)+ function5(f3_file)
}
```

As shown in Example 3, below, the second conventional approach is to create a library of shared functions and one or more scripts to combine the various functions.

### EXAMPLE 3

```
Library
function4(f1_results, f2_results) {
  return f1_results + f2_results
}
function5(f3_results) {
  return f3_results + 1
}
function6(f4_results, f5_results) {
  return f4_results + f5_results
}
Scripts
f1_results = function1(f1_filepath)
f2_results = function2(f2_filepath)
f3_results = function3(f3_filepath)
f4_results = function4(f1_results, f2_results)
f5_results = function5(f3_results)
f6_results = function6(f4_results, f5_results)
```

While the approach shown in Example 3 is less brittle than that of Example 2, it requires the creation and maintenance of scripts that are not easily reused. For example, if a user wants to introduce a new function (e.g., Function 7) that depends on Function 6, the user would either need to create a new script to aggregate all of the previous steps with the addition of Function 7, or they user would need to configure and employ orchestration software to combine multiple scripts. This solution is difficult to maintain, as any dependent scripts would need to propagate correct parameters to the original script.

Currently, a number of programs exist to allow users to create relatively simple workflows or pipelines to perform multi-part calculations. For example, workflow management applications, such as those offered by Knime.com AG, Alteryx Inc. and Integrify Inc., provide a user interface to allow users to manually create pipelines by connecting data sources, processing logic and output sources. Unfortunately, these applications allow users to only employ the conventional techniques shown in Examples 2 and 3, above, which are not suitable for handling the large-scale and complex pipelines required for precision medicine.

Accordingly, there is a need for data processing platforms that allow for the creation, management, and execution of user-defined, flexible pipelines that are capable of performing the complex calculations required for precision medicine. It would be beneficial if such platforms provided functionality to create reusable components that may be programmatically combined to form modular pipelines that may be reused and/or dynamically modified, as desired or required, for multiple datasets.

### SUMMARY

In accordance with the foregoing objectives and others, exemplary data processing platforms embodied in systems, computer-implemented methods, apparatuses and/or software applications are described herein. The described platforms allow for the creation and execution of user-defined, data-driven pipelines. Such pipelines may be associated with one or more connected data nodes, which define the location and type of data that a pipeline uses as input or output and the operations to be performed by the pipeline. In certain embodiments, the pipelines may be associated with node graphs, such as direct acyclic graphs (“DAGs”), which include any number of nodes connected together via dependency injection.

The pipelines employed by the described platforms may also be associated with context information, which specifies dataset-specific configurations and includes logic required to generate and execute the associated nodes. The context information may further include node substitution information that may be used in executing data from different data sources with different formats on generic pipelines that depend on standard input format. The context information may additionally or alternatively include logic that allows for caching of node output, data filtering, and/or dynamic node modification.

In one embodiment, a computer-implemented method is provided. The method may include, for example, receiving, by a computer, raw input data associated with a first format; storing, by the computer, the raw input data in a first memory; storing, by the computer, a plurality of data nodes, each of the data nodes adapted to receive an input and manipulate the input according to an associated functionality to generate an output; and/or storing, by a computer, a context object associated with a pipeline. The context object may include context information that is associated with one or more input nodes selected from the plurality of data nodes, the input nodes adapted to receive the raw input data stored in the first memory, and manipulate the raw input data according to the functionality associated with each of the input nodes to generate standardized data associated with a standardized format that is different than the first format; one or more processing nodes selected from the plurality of data nodes, the processing nodes adapted to receive the standardized data; manipulate the standardized data according to the functionality associated with each of the processing nodes to generate output data; and/or relationship information corresponding to how each of the input nodes is connected to one or more other input nodes, how at least one of the input nodes is connected to at least one of the processing nodes, and/or how each of the processing nodes is connected to one or more other processing nodes. The method may also include: receiving, by the computer, a data processing request associated with the pipeline and the raw input data; and, upon receiving the request: creating, by the computer, a node graph based on the context information, the node graph including the input nodes and the processing nodes, wherein at least one of the input nodes is linked to the first memory such that the raw input data is received therefrom, and wherein at least one of the processing nodes is linked to at least one of the input nodes such that the standardized data is received therefrom; processing, by the computer, the raw input data to the output data via the node graph; and/or storing, by the computer, the output data.

In another embodiment, a system including one or more processing units, and one or more processing modules is provided. The system may be configured by the one or more processing modules to: receive raw input data associated with a first format; store the raw input data in a first memory; and/or store a plurality of data nodes, each of the data nodes adapted to receive an input and manipulate the input according to an associated functionality to generate an output. The system may also be configured to store a context object associated with a pipeline, the context object including context information associated with (1) one or more input nodes selected from the plurality of data nodes, the input nodes adapted to: receive the raw input data stored in the first memory and manipulate the raw input data according to the functionality associated with each of the input nodes to generate standardized data associated with a standardized format that is different than the first format; (2) one or more processing nodes selected from the plurality of data nodes, the processing nodes adapted to: receive the standardized data, manipulate the standardized data according to the functionality associated with each of the processing nodes to generate output data; and/or (3) relationship information corresponding to: how each of the input nodes is connected to one or more other input nodes, how at least one of the input nodes is connected to at least one of the processing nodes, and/or how each of the processing nodes is connected to one or more other processing nodes. In certain embodiments, the system may be additionally configured by the processing modules to: receive a data processing request associated with the pipeline and the raw input data and, upon receiving the request: create a node graph based on the context information, the node graph including the input nodes and the processing nodes, wherein at least one of the input nodes is linked to the first memory such that the raw input data is received therefrom, and wherein at least one of the processing nodes is linked to at least one of the input nodes such that the standardized data is received therefrom; process the raw input data to the output data via the node graph; and store the output data.

In the above embodiment, the context information may also include one or more second input nodes selected from the plurality of data nodes, the second input nodes adapted to: receive second raw input data associated with a second format that is different than both the first format and the standardized format, and manipulate the second raw input data according to the functionality associated with each of the second input nodes to generate the standardized data. Moreover, the relationship information may further correspond to how each of the second input nodes is connected to one or more other second input nodes. Accordingly, the system may be further configured to receive the second raw input data; store the second raw input data in a second memory; receive a second data processing request associated with the pipeline and the second raw input data; and, upon receiving the second request: create a second node graph based on the context information, the second node graph including the second input nodes and the processing nodes, wherein at least one of the second input nodes is linked to the second memory such that the second raw input data is received therefrom, and wherein at least one of the processing nodes is linked to at least one of the second input nodes such that the standardized data is received therefrom; and/or process the second raw input data to the output data via the second node graph.

The details of one or more embodiments of the subject matter of this specification are set forth in the accompanying drawings and the description below. Other features, aspects, and advantages of the subject matter will become apparent from the description and the drawings.

### BRIEF DESCRIPTION OF THE DRAWINGS

FIG. 1 shows an exemplary system 100 according to an embodiment.

FIG. 2 shows an exemplary computing machine 200 and modules 250 according to an embodiment.

FIG. 3 shows an exemplary platform 300 configured to create and execute data processing pipelines according to an embodiment.

FIG. 4 shows an exemplary pipeline 401 comprising a node graph 410 and context information 405, wherein the pipeline is adapted to process input data (I41-I43).

FIG. 5 shows an exemplary node graph 500 comprising an output node N57 that depends on node N46 of FIG. 4.

FIG. 6 shows an exemplary pipeline 601 associated with context information 605 that includes node substitution information 606.

FIG. 7 shows an exemplary node graph 700 for preparing reports from patient records according to an embodiment.

FIG. 8 shows an exemplary method of filtering the node graph 700 of FIG. 7 according to a specified date variable.

FIG. 9 shows an exemplary node graph 900 having caching functionality according to an embodiment.

FIG. 10 shows an exemplary reports screen 1000 including demographic information 1003, patient history information 1004, patient comorbidities information 1005, patient claims information 1006, and diagnoses and procedures information 1007 according to an embodiment.

FIG. 11 shows an exemplary reports screen 1100 including financial information 1101, comorbidity cost information 1110, and patient cost information 1115 according to an embodiment.

FIG. 12 shows an exemplary reports screen 1200 including medications information 1201 according to an embodiment.

FIG. 13 shows an exemplary reports screen 1300 including lab tests information 1301 according to an embodiment.

FIG. 14 shows an exemplary method 1400 according to an embodiment.

FIG. 15 shows an exemplary risk reports screen 1500 according to an embodiment.

### DETAILED DESCRIPTION

Various systems, methods, and apparatuses are described herein that allow users to create and manage data processing pipelines comprising modular components. The disclosed embodiments provide a framework that empowers users to create highly dynamic units of work (i.e., nodes) that may be connected or otherwise combined to create flexible, maintainable and reusable data processing pipelines.

The platforms may be adapted to connect to various systems and databases in order to receive and store raw input data therefrom. For example, the platform may receive information from EHRs, insurance claims databases, health facility systems (e.g., systems associated with doctors' offices, laboratories, hospitals, pharmacies, etc.), and/or financial systems.

Upon receiving raw input data, the platform may execute one or more pipelines to process the raw input data into input information. Such processing may include, for example, cleaning, validating, and/or normalizing the raw input data into and storing the resulting input information in one or more databases.

In certain embodiments, the described platforms may employ one or more pipelines to monitor, analyze and generate reports relating to stored input information. For example, in the healthcare context, a pipeline may be employed to scan stored input information in order to determine patient demographics information, diagnoses and procedures information, medications information, lab tests information and/or financial information that is included in certain input information, and any problems or issues relating to such information. Such information may be output in the form of a downloadable file (i.e., a report) and/or may be displayed to a user via a visual interface (i.e., a dashboard).

Embodiments of the described platforms may also provide functionality to help organizations understand risk factors that lead to adverse events and to determine which users are at an increased risk of experiencing adverse events in the future. In the healthcare context, the platform may employ pipelines to search for patient information across stored input information, correlate patient information to specific patients, analyze such information to learn important risk factors for various adverse events, and/or to predict the likelihood that particular patients will experience such adverse events (e.g., via a risk score). The platform may output risk information, such as risk factors and patient risk scores, in the form of downloadable reports and/or online dashboards.

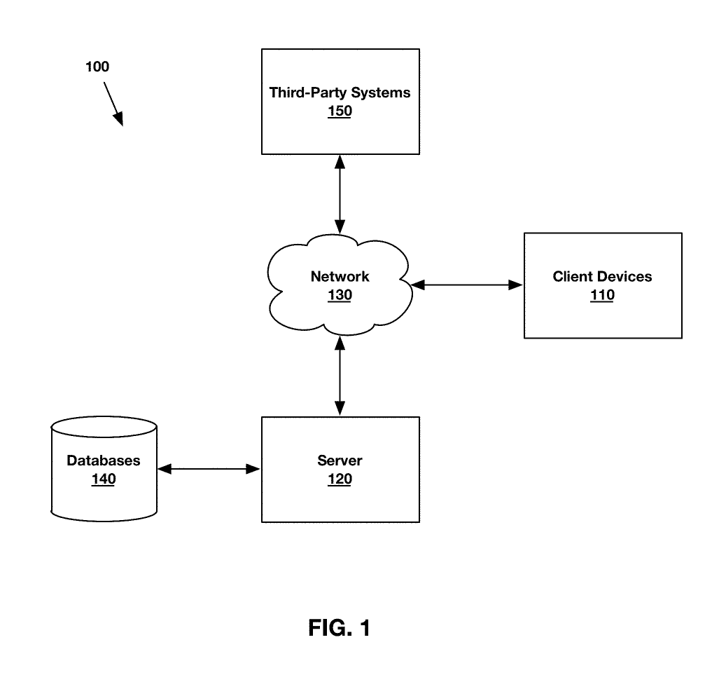

Referring to FIG. 1, a block diagram of an exemplary modular data processing pipeline system according to an embodiment 100 is illustrated. As shown, the system comprises any number of users accessing a server 120 via a network 130. In certain embodiments, a user may access the server 120 via a client device 110 connected to the network 130.

Generally, a client device 110 may be any device capable of running a client application and/or of accessing the server 120 (e.g., via the client application or via a web browser). Exemplary client devices 110 may include desktop computers, laptop computers, smartphones, and/or tablets.

The relationship of client 110 and server 120 arises by virtue of computer programs running on the respective computers and having a client-server relationship to each other. Accordingly, each of the client devices 110 may have a client application running thereon, where the client application may be adapted to communicate with a server application running on a server 120, for example, over a network 130. Thus, the client application and server 120 may be remote from each other. Such a configuration may allow users of client applications to input information and/or interact with the server from any location.

As discussed in detail below, a client application may be adapted to present various user interfaces to users. Such user interfaces may be based on information stored on the client device 110 and/or received from the server 120. Accordingly, the client application may be written in any form of programming language, including compiled or interpreted languages, or declarative or procedural languages, and it can be deployed in any form, including as a standalone program or as a module, component, subroutine, or other unit suitable for use in a computing environment. Such software may correspond to a file in a file system. A program can be stored in a portion of a file that holds other programs or data. For example, a program may include one or more scripts stored in a markup language document; in a single file dedicated to the program in question; or in multiple coordinated files (e.g., files that store one or more modules, sub programs, or portions of code).

The client application can be deployed and/or executed on one or more computer machines that are located at one site or distributed across multiple sites and interconnected by a communication network. In one embodiment, a client application may be installed on (or accessed by) one or more client devices 110. It will be apparent to one of ordinary skill in the art that, in certain embodiments, any of the functionality of a client may be incorporated into the server, and vice versa. Likewise, any functionality of a client application may be incorporated into a browser-based client, and such embodiments are intended to be fully within the scope of this disclosure. For example, a browser-based client application could be configured for offline work by adding local storage capability, and a native application could be distributed for various native platforms (e.g., Microsoft Windows™, Apple MacOS™, Google Android™ or Apple iOS™) via a software layer that executes the browser-based program on the native platform.

In one embodiment, communication between a client application and the server may involve the use of a translation and/or serialization module. A serialization module can convert an object from an in-memory representation to a serialized representation suitable for transmission via HTTP/HTTPS or another transport mechanism. For example, the serialization module may convert data from a native, in-memory representation into a JSON string for communication over the client-to-server transport protocol.

Similarly, communications of data between a client device 110 and the server 120 may be continuous and automatic, or may be user-triggered. For example, the user may click a button or link, causing the client to send data to the server. Alternately, a client application may automatically send updates to the server periodically without prompting by a user. If a client sends data autonomously, the server may be configured to transmit this data, either automatically or on request, to additional clients and/or third-party systems.

In certain embodiments, the server 120 and/or the client device 110 may be adapted to receive, determine, record and/or transmit application information. The application information may be received from and/or transmitted to the client application. Moreover, any of such application information may be stored in and/or retrieved from one or more local or remote databases (e.g., database 140).

Exemplary application information may include: user identification information (e.g., name, username or unique ID, password, contact information, billing information, user privileges information, etc.); contact information (e.g., email address, mailing address, phone number, etc.); billing information (e.g., credit card information, billing address, etc.); settings information; patient information (e.g., a unique ID, demographics information, diagnoses and procedures information, comorbidities information, medications information, lab tests information, insurance information); insurance claims information and/or various financial information.

In one embodiment, the server 120 may be connected to one or more third-party systems 150 via the network 130. Third-party systems 150 may store information in one or more databases that may be accessed by the server. Exemplary third-party systems may include, but are not limited to: electronic medical records (“EMR”) storage systems, biometric devices and databases storing biometric device data, systems storing patient survey data, and/or systems that store and/or manage insurance claims data. Other exemplary third-party systems may include: payment and billing systems, contact management systems, customer relationships management systems, and/or cloud-based storage and backup systems.

The server 120 may be capable of retrieving and/or storing information from third-party systems 150, with or without user interaction. Moreover, the server may be capable of transmitting stored and/or generated information to third-party systems.

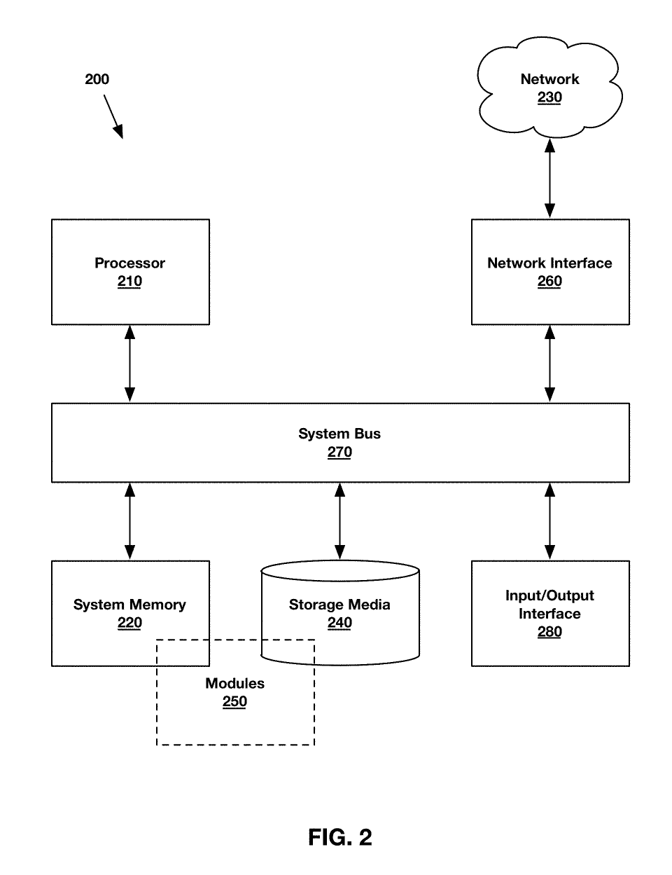

Referring to FIG. 2, a block diagram is provided illustrating a computing machine 200 and modules 250 in accordance with one or more embodiments presented herein. The computing machine 200 may correspond to any of the various computers, servers, mobile devices, embedded systems, or computing systems presented herein (e.g., the client device(s) 110, server(s) 120, and/or third-party system(s) 150 of FIG. 1). The modules 250 may comprise one or more hardware or software elements configured to facilitate the computing machine 200 in performing the various methods and processing functions presented herein.

The computing machine 200 may comprise all kinds of apparatuses, devices, and machines for processing data, including but not limited to, a programmable processor, a computer, and/or multiple processors or computers. For example, the computing machine 200 may be implemented as a conventional computer system, an embedded controller, a laptop, a server, a mobile device, a smartphone, a set-top box, over-the-top content TV (“OTT TV”), Internet Protocol television (“IPTV”), a kiosk, a vehicular information system, one more processors associated with a display, a customized machine, any other hardware platform and/or combinations thereof. Moreover, a computing machine may be embedded in another device, such as but not limited to, a personal digital assistant (“PDA”), a smartphone, a tablet, or a portable storage device (e.g., a universal serial bus (“USB”) flash drive). In some embodiments, the computing machine 200 may be a distributed system configured to function using multiple computing machines interconnected via a data network or system bus 270.

As shown, an exemplary computing machine 200 may include various internal and/or attached components, such as a processor 210, system bus 270, system memory 220, storage media 240, input/output interface 280, and network interface 260 for communicating with a network 230.

The processor 210 may be configured to execute code or instructions to perform the operations and functionality described herein, manage request flow and address mappings, and to perform calculations and generate commands. The processor 210 may be configured to monitor and control the operation of the components in the computing machine 200. The processor 210 may be a general-purpose processor, a processor core, a multiprocessor, a reconfigurable processor, a microcontroller, a digital signal processor (“DSP”), an application specific integrated circuit (“ASIC”), a graphics processing unit (“GPU”), a field programmable gate array (“FPGA”), a programmable logic device (“PLD”), a controller, a state machine, gated logic, discrete hardware components, any other processing unit, or any combination or multiplicity thereof. The processor 210 may be a single processing unit, multiple processing units, a single processing core, multiple processing cores, special purpose processing cores, coprocessors, or any combination thereof. In addition to hardware, exemplary apparatuses may comprise code that creates an execution environment for the computer program (e.g., code that constitutes one or more of: processor firmware, a protocol stack, a database management system, an operating system, and a combination thereof). According to certain embodiments, the processor 210 and/or other components of the computing machine 200 may be a virtualized computing machine executing within one or more other computing machines.

The system memory 220 may include non-volatile memories such as read-only memory (“ROM”), programmable read-only memory (“PROM”), erasable programmable read-only memory (“EPROM”), flash memory, or any other device capable of storing program instructions or data with or without applied power. The system memory 220 also may include volatile memories, such as random-access memory (“RAM”), static random-access memory (“SRAM”), dynamic random-access memory (“DRAM”), and synchronous dynamic random-access memory (“SDRAM”). Other types of RAM also may be used to implement the system memory. The system memory 220 may be implemented using a single memory module or multiple memory modules. While the system memory is depicted as being part of the computing machine 200, one skilled in the art will recognize that the system memory may be separate from the computing machine without departing from the scope of the subject technology. It should also be appreciated that the system memory may include, or operate in conjunction with, a non-volatile storage device such as the storage media 240.

The storage media 240 may include a hard disk, a compact disc read only memory (“CD-ROM”), a digital versatile disc (“DVD”), a Blu-ray disc, a magnetic tape, a flash memory, other non-volatile memory device, a solid-state drive (“SSD”), any magnetic storage device, any optical storage device, any electrical storage device, any semiconductor storage device, any physical-based storage device, any other data storage device, or any combination/multiplicity thereof. The storage media 240 may store one or more operating systems, application programs and program modules such as module, data, or any other information. The storage media may be part of, or connected to, the computing machine 200. The storage media may also be part of one or more other computing machines that are in communication with the computing machine such as servers, database servers, cloud storage, network attached storage, and so forth.

The modules 250 may comprise one or more hardware or software elements configured to facilitate the computing machine 200 with performing the various methods and processing functions presented herein. The modules 250 may include one or more sequences of instructions stored as software or firmware in association with the system memory 220, the storage media 240, or both. The storage media 240 may therefore represent examples of machine or computer readable media on which instructions or code may be stored for execution by the processor. Machine or computer readable media may generally refer to any medium or media used to provide instructions to the processor. Such machine or computer readable media associated with the modules may comprise a computer software product. It should be appreciated that a computer software product comprising the modules may also be associated with one or more processes or methods for delivering the module to the computing machine via the network, any signal-bearing medium, or any other communication or delivery technology. The modules 250 may also comprise hardware circuits or information for configuring hardware circuits such as microcode or configuration information for an FPGA or other PLD.

The input/output (“I/O”) interface 280 may be configured to couple to one or more external devices, to receive data from the one or more external devices, and to send data to the one or more external devices. Such external devices along with the various internal devices may also be known as peripheral devices. The I/O interface 280 may include both electrical and physical connections for operably coupling the various peripheral devices to the computing machine 200 or the processor 210. The I/O interface 280 may be configured to communicate data, addresses, and control signals between the peripheral devices, the computing machine, or the processor. The I/O interface 280 may be configured to implement any standard interface, such as small computer system interface (“SCSI”), serial-attached SCSI (“SAS”), fiber channel, peripheral component interconnect (“PCP”), PCI express (PCIe), serial bus, parallel bus, advanced technology attachment (“ATA”), serial ATA (“SATA”), universal serial bus (“USB”), Thunderbolt, FireWire, various video buses, and the like. The I/O interface may be configured to implement only one interface or bus technology. Alternatively, the I/O interface may be configured to implement multiple interfaces or bus technologies. The I/O interface may be configured as part of, all of, or to operate in conjunction with, the system bus 270. The I/O interface 280 may include one or more buffers for buffering transmissions between one or more external devices, internal devices, the computing machine 200, or the processor 210.

The I/O interface 280 may couple the computing machine 200 to various input devices including mice, touch-screens, scanners, biometric readers, electronic digitizers, sensors, receivers, touchpads, trackballs, cameras, microphones, keyboards, any other pointing devices, or any combinations thereof. When coupled to the computing device, such input devices may receive input from a user in any form, including acoustic, speech, visual, or tactile input.

The I/O interface 280 may couple the computing machine 200 to various output devices such that feedback may be provided to a user via any form of sensory feedback (e.g., visual feedback, auditory feedback, or tactile feedback). For example, a computing device can interact with a user by sending documents to and receiving documents from a device that is used by the user (e.g., by sending web pages to a web browser on a user's client device in response to requests received from the web browser). Exemplary output devices may include, but are not limited to, displays, speakers, printers, projectors, tactile feedback devices, automation control, robotic components, actuators, motors, fans, solenoids, valves, pumps, transmitters, signal emitters, lights, and so forth. And exemplary displays include, but are not limited to, one or more of: projectors, cathode ray tube (“CRT”) monitors, liquid crystal displays (“LCD”), light-emitting diode (“LED”) monitors and/or organic light-emitting diode (“OLED”) monitors.

Embodiments of the subject matter described in this specification can be implemented in a computing machine 200 that includes one or more of the following components: a backend component (e.g., a data server); a middleware component (e.g., an application server); a frontend component (e.g., a client computer having a graphical user interface (“GUI”) and/or a web browser through which a user can interact with an implementation of the subject matter described in this specification); and/or combinations thereof. The components of the system can be interconnected by any form or medium of digital data communication, such as but not limited to, a communication network.

Accordingly, the computing machine 200 may operate in a networked environment using logical connections through the network interface 260 to one or more other systems or computing machines across the network 230. The network 230 may include wide area networks (“WAN”), local area networks (“LAN”), intranets, the Internet, wireless access networks, wired networks, mobile networks, telephone networks, optical networks, or combinations thereof. The network 230 may be packet switched, circuit switched, of any topology, and may use any communication protocol. Communication links within the network 230 may involve various digital or an analog communication media such as fiber optic cables, free-space optics, waveguides, electrical conductors, wireless links, antennas, radio-frequency communications, and so forth.

The processor 210 may be connected to the other elements of the computing machine 200 or the various peripherals discussed herein through the system bus 270. It should be appreciated that the system bus 270 may be within the processor, outside the processor, or both. According to some embodiments, any of the processor 210, the other elements of the computing machine 200, or the various peripherals discussed herein may be integrated into a single device such as a system on chip (“SOC”), system on package (“SOP”), or ASIC device.

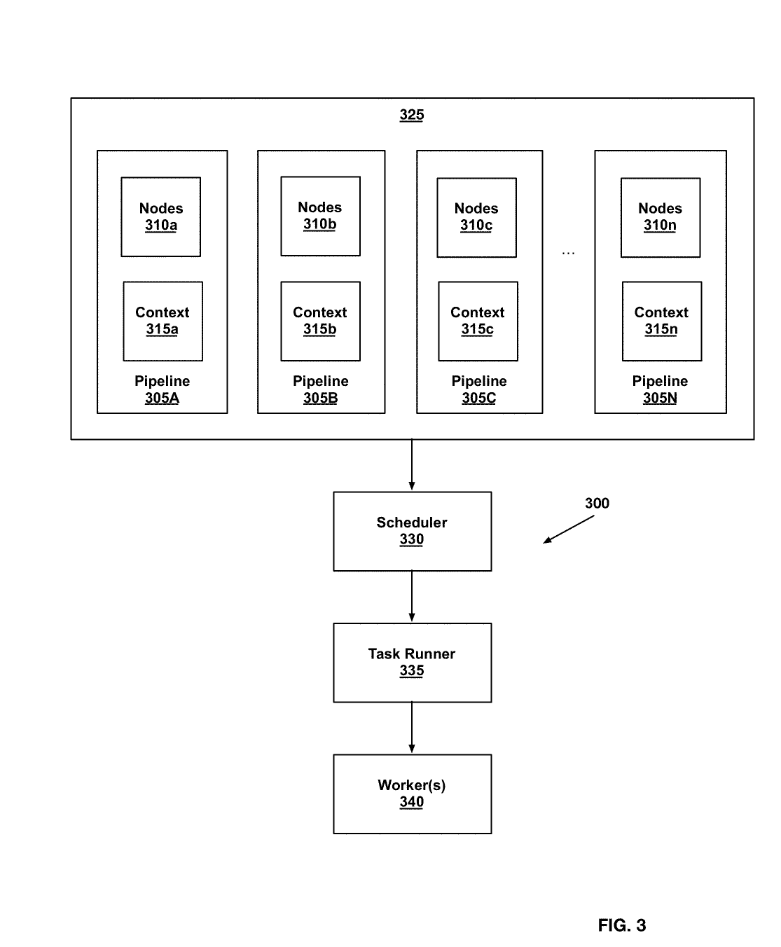

Referring to FIG. 3, an exemplary platform 300 configured to create and execute flexible, maintainable and reusable data processing pipelines is illustrated. The platform may include any number of pipelines (305a, 305b, 305c through 305n) (referred to herein as “pipelines 305” for convenience) stored in an internal or external memory 325. As shown, each of the pipelines 305 may be associated with any number of nodes (310a, 310b, 310c through 310n) (referred to herein as “nodes 310” for convenience) and context information (315a, 315b, 315c through 315n) (referred to herein as “context information 315 for convenience”). Such pipelines 305, nodes 310 and context information 315 may be created graphically via a user interface, textually by providing a source code file, and/or programmatically via a software development kit (“SDK”) or an application programming interface (“API”).

Generally, each of the nodes 310 may comprise a dynamic unit of work that may be connected to, or otherwise combined with, other nodes to create modular data processing pipelines. To that end, each node 310 may be associated with one or more of the following: input or dependency information (e.g., a location and type of input data to be received by the node), output or results information (e.g., a location and type of output data to generated by the node), logic or computational aspects to manipulate input data, scheduling information, a status, and/or a timeout value. It will be appreciated that data nodes 310 can inherit properties from one or more parent nodes, and that relationships among nodes may be defined by reference.

The context information 315 typically includes input information corresponding to the location of each input source to the pipeline 305, dependency or relationship information corresponding to how each of the nodes in the pipeline should be connected, and execution information including the necessary logic to execute each of the nodes. As discussed in detail below, context information 315 may further comprise node substitution information, modifier information, and/or caching information to provide novel and powerful data processing functionality.

The platform 300 may include various components to manage and execute pipelines 305, such as a task scheduler 330, a task runner 335 and/or one or more computing resources 340 (i.e., workers). Generally, these components work together to execute the pipelines 305 by (1) compiling the various pipeline components (i.e., data nodes 310 and context information 315), (2) creating a set of actionable tasks, (3) scheduling the tasks, and/or (4) assigning such tasks to a computational resource.

In one embodiment, the scheduler 330 splits operations into a plurality of tasks, wherein each task is associated with at least one input node and at least one output node, and wherein each task comprises a complete definition of work to be performed. As discussed in detail below, exemplary tasks may include data manipulations such as, but not limited to, joins (an operation performed to establish a connection between two or more database tables, thereby creating a relationship between the tables), filters (a program or section of code that is designed to examine each input or output request for certain qualifying criteria and then process or forward it accordingly), aggregations (a process in which information is gathered and expressed in a summary form for purposes such as statistical analysis), caching (i.e., storing results for later use), counting, renaming, searching, calculating a value, determining a maximum, determining a minimum, determining a mean, determining a standard deviation, sorting, and/or other table operations.

The scheduler 330 may also determine scheduling information for each of the tasks in order to specify when a given task should be executed by a worker. For example, tasks may be scheduled to run: on activation, periodically (i.e., at the beginning or end of a predetermined period of time), at a starting time and date, and/or before an ending time and date.

The scheduler 330 may then provide a complete set of tasks and corresponding scheduling information to one or more task runners 335 for processing. Generally, task runners 335 are applications that poll a data pipeline for scheduled tasks and then execute those tasks on one or more machines (workers) 340. When a task is assigned to a task runner 335, it performs the task and reports its status back to the data pipeline.

It will be appreciated that, in certain embodiments, the execution of computations may be “lazy,” such that the organization of nodes can be performed without executing the nodes until explicitly instructed later. It will be further appreciated that, in some embodiments, the platform 300 may be agnostic to lower-level computational scheduling that formulates and allocates tasks among computational resources. That is, the platform may employ one or more third-party systems to schedule and execute low-level data manipulations, such as a single computing machine or a distributed clusters of computing machines running Apache Spark and/or Apache Hadoop.

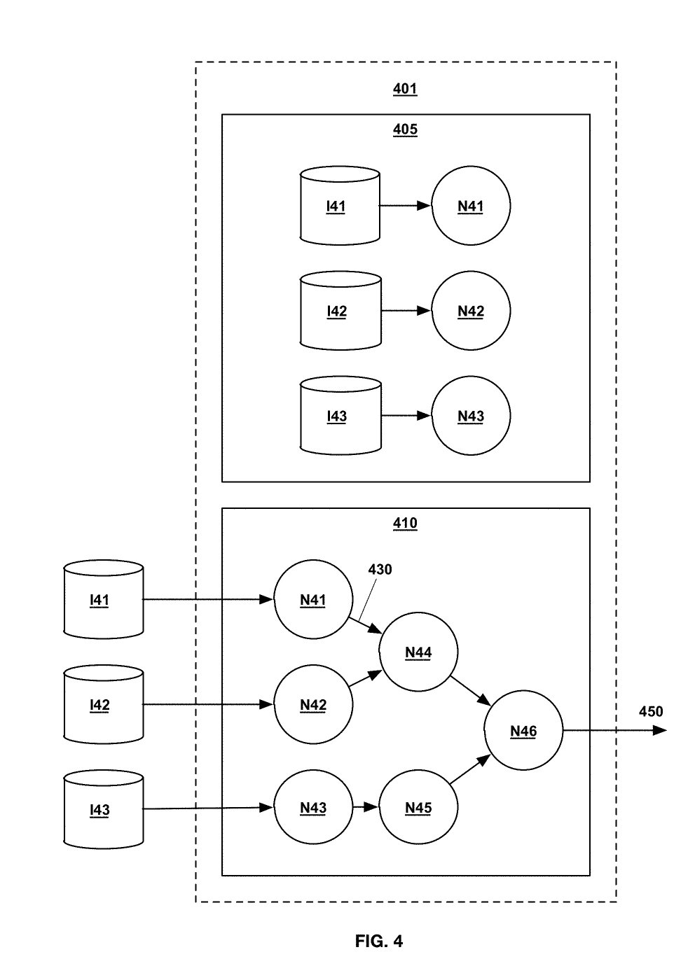

Referring to FIG. 4, an exemplary pipeline 401 comprising a node graph 410 and corresponding context information 405 is illustrated. Generally, the platform may employ pipelines comprising node graphs, such as DAGs in order to solve the parameter propagation issues of conventional data pipelines (discussed above in reference to Examples 1-3). Specifically, such node graphs facilitate maintenance and reuse of existing nodes and creation of new nodes, because each node in the graph is loosely coupled to one or more other nodes through dependency injection.

As shown, the node graph 410 comprises a plurality of data nodes (N41-N46) chained together via dependency. In such configuration, node N44 will perform some computation on the results of nodes N41 and N42; node N45 will perform some computation on the results of node N43; and node N46 will perform some computation on the results of nodes N44 and N45. Accordingly, execution of the pipeline will return a result 450 that is equal to the output of node N46.

The pipeline 401 may also be associated with context information 405, which may include the location of each input source (I41-I43), the logic required to generate the node graph 410 from the earliest node(s) (N41-N43) to the ending node (N46), and the necessary logic to execute each of the nodes (N41-N46) in the node graph. The platform may thus employ a higher-level node graph to construct and orchestrate lower-level computational node graphs. The higher-level graph composes and orchestrates, in a parsimonious fashion, multiple computational aspects, such as caching of intermediate calculations, various filtering patterns, and complex data transformations that would otherwise be difficult to express and optimize.

In the illustrated embodiment, the context information 405 specifies that node N41 will receive data from input source I41; node N42 will receive data from input source I42; and node N43 will receive data from input source I43. Accordingly, node N46 may be executed with the configured context information 405, which will create the node graph 410, and the N41, N42 and N43 nodes will load their data from the correct input sources (i.e., I41, I42 and I43, respectively).

An important aspect of this approach is that node N46 does not need to propagate the input file arguments down the dependency chain (i.e., to nodes N41, N42 and/or N43). This is a significant improvement over conventional pipelines, which require multiple functions to be modified to add more arguments (see Example 2, above). Moreover, this approach provides a low-cost solution to achieve decoupling, as the configuration information 405 may only need to be set once for each new input source (i.e., each new input dataset schema).

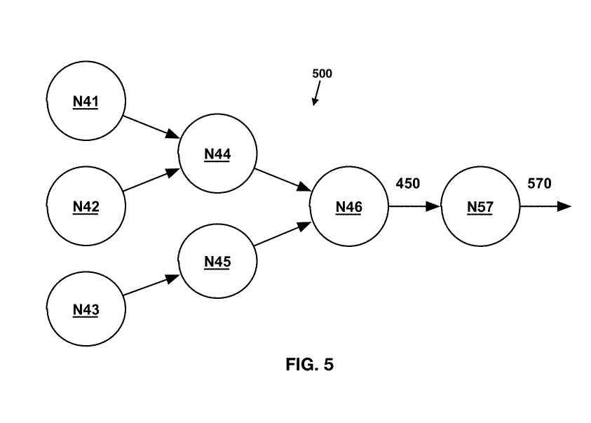

For example, FIG. 5, illustrates a newly created node N57 that depends on the results of node N46 in FIG. 4. Example 4, below, illustrates exemplary pseudocode for creating new node N57. The pseudocode executes a method (“get_results”) that receives the results 450 from node N46 as input data and performs additional processing on such input data to calculate an output 570. As shown, the new node N57 may be created and added to node N46 without knowing how the results of node N46 are calculated and/or the other nodes upon which node N46 depends.

### EXAMPLE 4

```
class N47(ComputationNode):
  def _init_(self):
    self._dep = N46( )
  def get_results(N46_results):
    return N46_results + 10
```

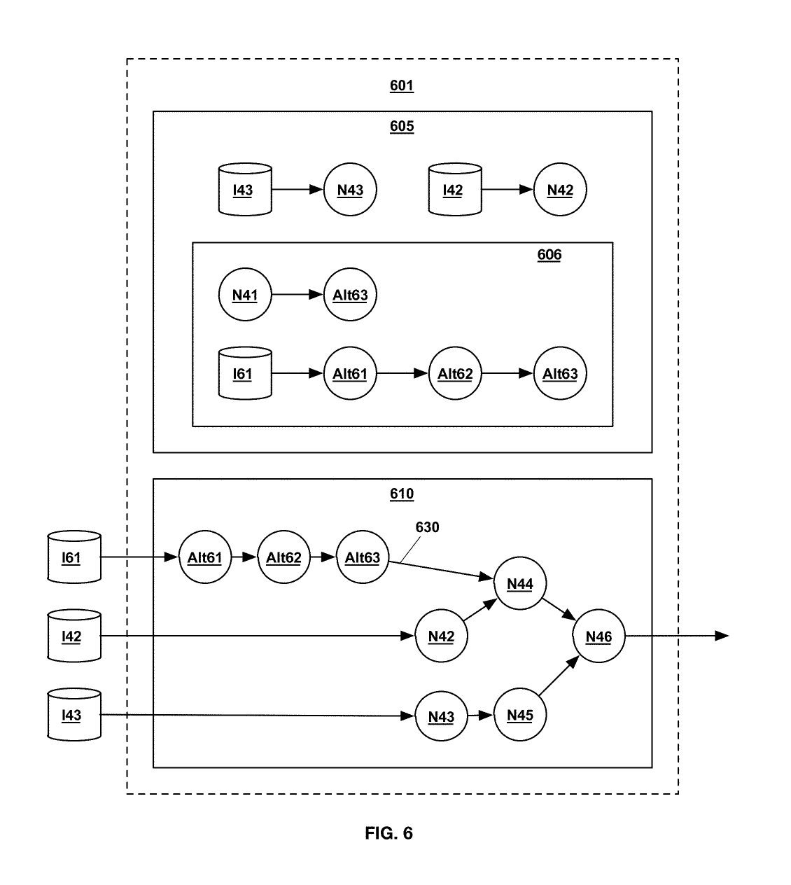

Referring to FIG. 6, an exemplary pipeline 601 comprising context information 605 that includes node substitution information 606 is illustrated. Generally, node substitution is a way to replace an original node (i.e., a “target node”) with one or more new nodes (i.e., “substitute nodes”). Node substitution is useful, for example, in executing different data sources with different formats on a single generic pipelines that depends on standard input format.

In the illustrated embodiment, substitute nodes Alt61, Alt62, Alt63 represent nodes that are adapted to process data from dataset I61 into a standard or normalized format for use with the node N46 of FIG. 4. That is, the results/output 630 from the configuration of nodes Alt61, Alt62 and Alt63 will be in the same format as the results/output 430 from node N41 in FIG. 4.

As shown, context information 605 is provided with node substitution information 606 that instructs the program to substitute node Alt63 for node N41 when receiving input from dataset I61. Accordingly, when input from dataset I61 is to be used with the node graph 410 of FIG. 4 (i.e., when node N46 is executed with such input data), the system may create a new node graph 610 by replacing a target node (e.g., node N41 in FIG. 4) with one or more substitute nodes (e.g., node Alt63).

In order to utilize this approach, a user may first create one or more substitute nodes adapted to process input data to a particular format. And then the user may add node substitution information to a context information object, wherein the node substitution information includes the substitute nodes and a target node to be replaced by the substitute nodes. It will be appreciated that this process may only need to be completed once per dataset schema.

One benefit of the above-described technique is that it does not require client-specific aggregation code to allow a given pipeline to work with multiple datasets. For example, node N46 may be decoupled from all dataset-specific code, making it maintainable and reusable across datasets (e.g., both dataset I41 in FIG. 4 and dataset I61 in FIG. 6).

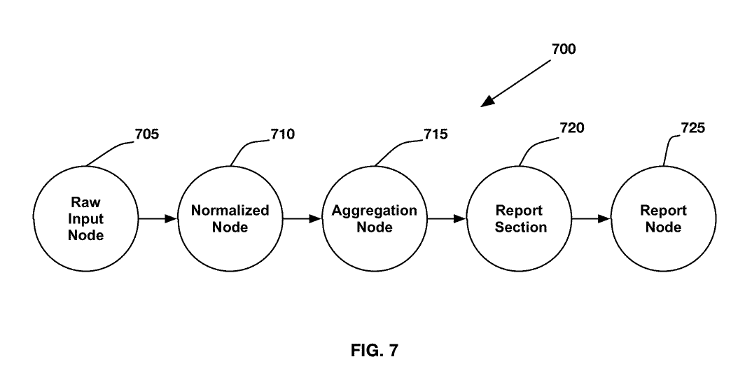

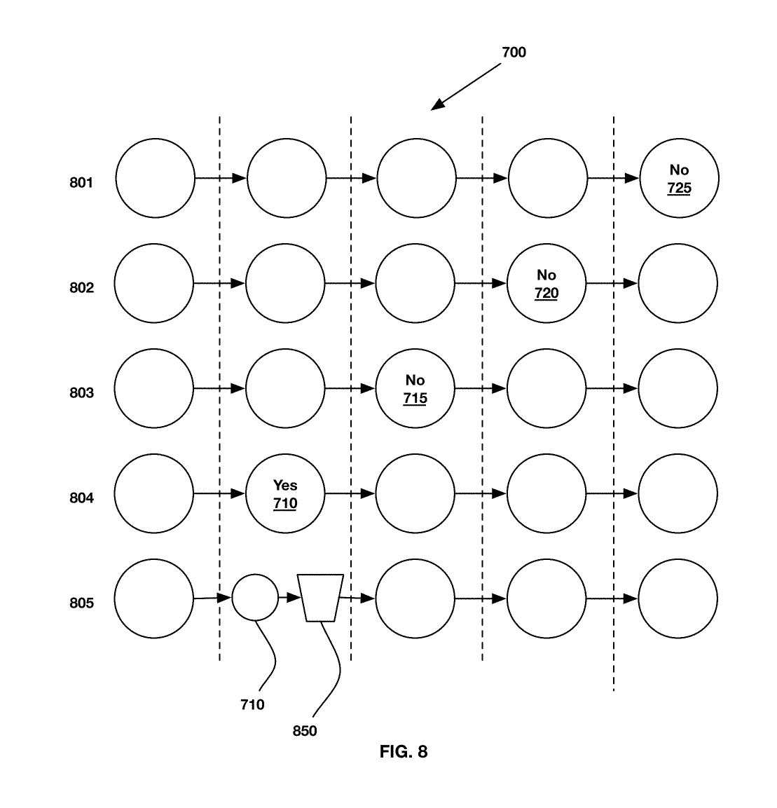

Referring to FIGS. 7-8, in some embodiments, the platform may employ one or more modifiers to alter a node graph at a given point, without requiring parameters to be added through the dependency chain. Such modifiers may allow for the creation of flexible pipelines that are easily modifiable at any point along the associated node graph.

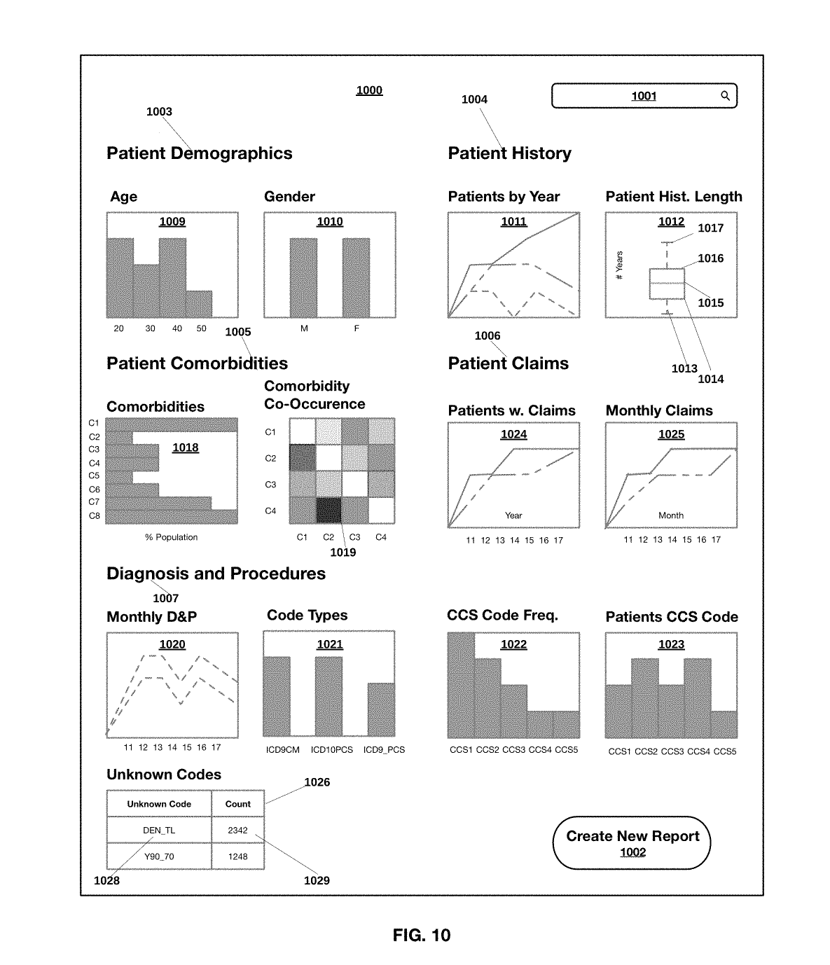

Referring to FIG. 7, an exemplary node graph 700 for preparing reports from patient records is illustrated. As shown, the node graph comprises a raw input node 705 to receive raw input data, a normalized node 710 to process the received input data into a standardized format, an aggregation node 715 to aggregate diagnoses and procedures records for each unique patient ID found in the normalized patient data, a report section node 720 to generate a section of a report and a report node 725 to generate a particular interface element to display information determined from the aggregated patient data (e.g., a table, chart or graph). In the illustrated embodiment, the report node 725 will generate a report comprising various summary information for all input data stored in the system. Exemplary reports are discussed in detail below in reference to FIGS. 10-13 and 15.

When creating reports for healthcare data, it is often necessary to filter the input data by one or more variables, such as a particular patient demographic, lab test, medical diagnosis, medical procedure, medication, comorbidity, and/or a specific time period (e.g., diagnoses that occurred in 1996). Unfortunately, pipelines may include certain nodes that remove important information when processing data, resulting in an inability to apply necessary filters. For example, the aggregation node 715 counts events in a time range and produces an output that does not include any date information that exists in the original input data received by node 705.

In such cases, conventional systems require either the addition of new date parameters throughout the entire dependency chain (see, e.g., Example 2), or the creation of a script to glue together pieces of logic (see, e.g., Example 3). As discussed above, both approaches tend to be repetitive and error-prone.

In stark contrast to such conventional systems, embodiments of the data processing platform employ a unique modifier approach that allows for node graphs to be modified at designated nodes, while keeping the remaining node graph structure intact. Modifiers work around the above parameter propagation restrictions by allowing for modification requests to be received by individual nodes after construction of a node graph and further allow for such requests to be handled by the context information. The modification request may be performed with a method contained in the context information. The method traverses the node graph backwards from the end node and asks each node whether it can respond to the request in a way that would make the graph fulfil the request. When a node in the node graph is capable of fulfilling the request (i.e., providing necessary information relating to the original input data), the system may automatically modify the graph as required to ensure the output of the graph fulfills the modification request.

FIG. 8 illustrates an exemplary method of modifying the node graph 700 of FIG. 7 to backfill a date variable that has been removed by the aggregation node 715 during report generation. As shown, each node in the node graph 700 is probed in sequence as to whether it can provide the required information (i.e., a date variable) for the graph to produce an output that is filtered by a specific date range. At step 801, the report node 725 is probed and responds with a “no” because it is located after the aggregation node 715 and so its output does not include a date variable. At step 802, the report section node 720 is probed and responds with a “no” for the same reason. And at step 803, the aggregation node 715 is probed and responds with a “no” because its output does not include a date variable.

At step 804, the normalized node 710 is probed and responds with a “yes” because it is located before the aggregation node 715 and so its output does include a date variable that may be used to satisfy the modification request. As such, at step 805, a modifier node 850 is added to the node graph 700 such that it depends from the normalized node 710. In the illustrated embodiment, the modifier node 850 is adapted to receive output from the normalized node 710 and to apply the modification request to such output (i.e., to filter the output according to the desired date range). Generally, modifier nodes 850 may be employed for many scenarios, including but not limited to: filtering, partitioning, obfuscating information and others.

It will be appreciated that nodes may work with modifiers by implementing a simple method, “get_mutator_for_modifier,” that returns an object that will mutate the node graph if the node can respond to the modifier. Most nodes will not implement this method, and the ones that do will often inherit the desired behavior from a mix-in class.

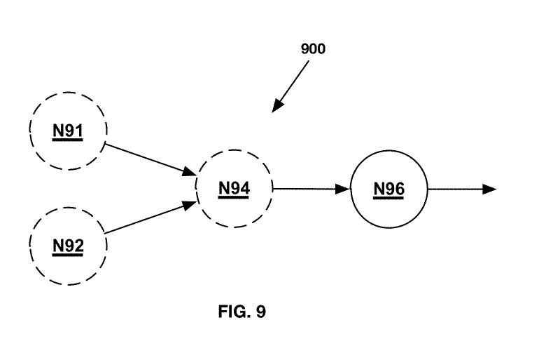

Referring to FIG. 9, an exemplary node graph 900 having caching functionality is illustrated. In certain embodiments, the system may be adapted to cache (i.e., store) output information of one or more nodes in a graph to be used as input data for other nodes. Such caching allows the system to compute output information for downstream nodes, based on the input data, without having to recalculate the previously-cached output information. In other words, the system does not need to perform the same calculation multiple times for a given input source.

As shown, calculations for node N94 have been cached by the system, wherein the cached calculations are represented with dashed lines around the nodes. Accordingly, when node N96 requests output information from node N94, the output information will simply be retrieved from a file. Accordingly, the system does not have to compute output information for nodes N91, N92 and N94 when determining the results of node N96.

In certain embodiments, the original node graph 900 may be modified (as discussed above) while traversing backwards from node N96 at the point where cached data will be used. Such modification may be automatically handled by a context information object.

In some embodiments, logic can be introduced to handle multiple modifiers. For example, one may desire date modification where some nodes encounter the cached node N94 shown in FIG. 9 that was previously cached in a narrower date range. If the date modifier is outside the date range of the cached node, then the system will check for this, and then continue back-traversal because the intent is to retrieve data outside of the date range of the previously cached node. However the cached node may still continue to be used or modified within the original date range of the cached node.

Referring to FIGS. 10-13 exemplary reports screens (1000, 1100, 1200, 1300) are illustrated. The reports screens display various summary information, which may be determined by employing the above-described pipelines to clean, normalize, and/or analyze input data from any number of data sources. As shown, such summary information may comprise statistics or analytics relating to patient demographic information 1003, patient history information 1004, patient comorbidities information 1005, patient claims information 1006, diagnoses and procedures information 1007, financial information 1101, medication information 1201, and/or lab tests information 1301. Summary information may be determined for each individual patient and/or across an entire patient population or a subset thereof. Similarly, the summary information may be determined for one or more time periods of any length.

Upon determining summary information from input data, the platform may save the information in one or more databases. The system may also provide the summary information to one or more users, for example, via one or more user interface screens of a client application, an API, and/or via creation of digital reports that may be stored, printed and/or displayed.

In certain embodiments, the platform may include a client application adapted to employ pipelines to determine summary information and to provide the same to users via one or more screens (e.g., 1000, 1100, 1200, 13000) comprising various user interface elements (e.g., graphs, charts, tables, lists, text, images, etc.). The user interface elements may be viewed and manipulated (e.g., filtered, sorted, searched, zoomed, positioned, etc.) by a user in order to understand insights about the input data.

The various summary information generated/displayed by the platform may be predetermined or may be customized by a user. For example, the client application may provide searching functionality 1001 to allow users to search for particular summary information and/or report-generating functionality 1002 to create custom reports comprising selected summary information. Such reports (e.g., 1000, 1100, 1200, 13000) may be in the form of web pages having a unique URL that may be accessed and/or shared. Alternatively, such reports may be in the form of a digital file that may be saved and/or shared.

As shown in FIG. 10, an exemplary reports screen 1000 may include demographics information 1003 for a plurality of unique patient records. The reports screen may show a breakdown of patients by, for example, race, gender 1010, marital status, current age 1009 and/or age at time of medical records. Such information may be shown across an entire patient population (i.e., all unique patient IDs found within the input data) or may be limited to information about patients that satisfy one or more specified criteria (e.g., patients with at least one diagnosis, procedure or medication claim in the past year).

Patient history information 1004 may also be determined and displayed. For example, a chart may display the number of “active” patients in each year 1011 (i.e., patients associated with at least one diagnosis, procedure, medication, lab test or claim in the respective year), the number of new active patients in each year and/or the total number of active patients throughout time. As another example, information relating to how many years' worth of data exists for each patient (i.e., patient history length) 1012 may also be provided. Generally, a patient's history length may be determined via a pipeline that includes one or more nodes to calculate the length between a date of the patient's first recorded event and a date of the patient's last record event. As shown, a patient history length chart may show a minimum 1013, a maximum 1017, a median 1015, a 25th percentile 1014, and a 75th percentile 1016 patient history length across a patient population.

In one embodiment, the reports screen 1000 may include patient comorbidities information 1005. As shown, a chart 1018 may provide information relating to the number of patients (or patient population percentage) associated with any number of comorbidities over a given time period. Additionally, a heatmap 1019 may also be provided to show how often patients are associated with specific pairs of comorbidities. It will be appreciated that, although any comorbidities may be included in reports, certain embodiments may limit reporting to comorbidities that are included in the Elixhauser Comorbidity Index, which is described in detail in Elixhauser A., et al. “Comorbidity measures for use with administrative data,” Med. Care 36:1 (1998) pp. 8-27, incorporated by reference herein in its entirety.

The reports screen 1000 may include various user interface elements relating to diagnoses and procedures information 1007 contained in the input data. As shown, diagnoses and procedures code types 1021 found within the input data may be determine and displayed, along with corresponding information, such as the total number of each code type found in each month or year and/or the total number of each code type found over a predefined period of time. Exemplary diagnosis and procedure code types may include any of the various International Classification of Diseases (ICD) codes, such as ICDA-8, ICD-9, ICD-9-CM, ICD-O (Oncology), ICD-10 and ICD-10-CA (Canadian Enhancements), ICD-9-PCS, and ICD10-PCS. The ICD coding method is described in detail in “International Statistical Classification of Diseases and Related Health Problems 10th Revision,” Geneva: World Health Organization, 2016; Quan, Hude et al., “Coding Algorithms for Defining Comorbidities in ICD-9-CM and ICD-10 Administrative Data,” Med. Care 43:11 (2005) pp. 1130-1139; and the Centers for Disease Control and Prevention (National Center for Health Statistics) website, available at cdc.gov/nchs/icd/. Each of the above references is incorporated by reference herein in its entirety.

In one embodiment, the system may employ pipelines to map each of the diagnoses and procedures codes found in the input data to a corresponding Clinical Classification Software (“CCS”) code in order to group events into a manageable number of clinically meaningful categories for exploration. Upon such mapping, the system may determine and display the total count of each CCS code 1022 over a given time period and/or the total number of patients (or percentage of patient population) associated with each CCS code 1023. It will be appreciated that such information may be determined and/or displayed for one or more levels of CCS codes (e.g., level 1, level 2, level 3 and/or level 4). CCS Codes are described in detail at the Health Cost and Utilization Project (“HCUP”) web site, available at hcup-us.ahrq.gov/toolssoftware/ccs/ccs.jsp.

The reports screen 1000 may further display patient claims information 1006. For example, one or more charts may display the total number of patients associated with at least one claim 1024 in a given time period (e.g., a month, a year, etc.). As another example, one or more charts may display the total number of claims 1025 that occurred during a given time period. These charts and/or others may further specify whether partial or full payment was received for each of the claims.

In certain embodiments, the reports screen 1000 may include a user interface element relating to unknown codes found in the input data. For example, a table 1026 may display any unknown diagnoses and procedures codes 1028 found in the input data along with the total number of occurrences 1029 of each unknown code over a given time period. As another example, a graph may display the total number of each unknown code found in each month or year and/or an aggregate total of unknown codes found in each month or year.

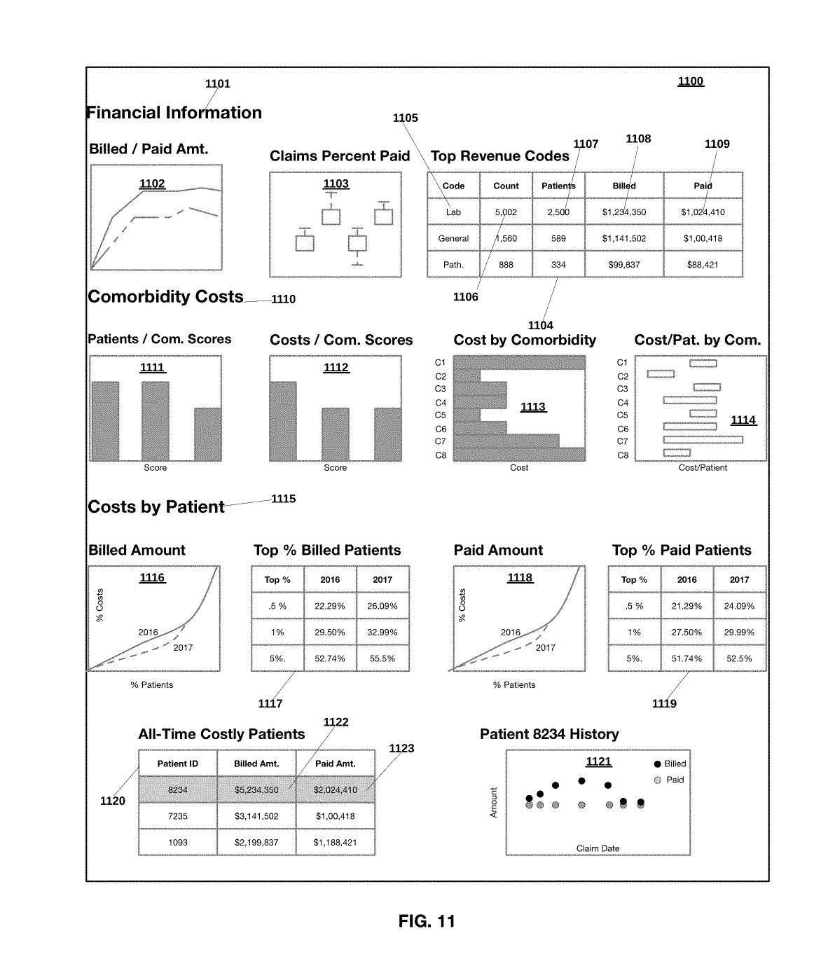

Referring to FIG. 11, an exemplary reports screen 1100 showing financial information 1101 determined from input data via one or more pipelines is illustrated. As shown, user interface elements may be included to display information relating to amounts billed, payments received, and costs. Such interface elements may display, for example: the total amount billed in a given time period 1102, total payments received in a given time period 1102, the percentage of amount billed that was paid 1103, the mean amount billed in a given time period and/or the mean amount paid in a given time period.

Information about revenue codes found in the input data may also be displayed via the reporting screen 1100. For example, each revenue code may be listed in a table 1104 along with corresponding information, such as a label 1105, the total number of times the revenue code was found in the data 1106, the total number of payments received for the revenue code, the total number of patients associated with the revenue code 1107, the maximum amount billed for the revenue code, the mean amount billed for the revenue code, the total amount billed for the revenue code 1108, the maximum payment received for the revenue code, the mean payment amount received for the revenue code, the total payment amount received for the revenue code 1109, an amount paid to amount billed ratio, and/or a difference between the amount billed and the amount paid for the revenue code. Although not shown, various scatter plots may be generated and displayed, including those showing: mean billed amount by revenue code frequency, mean billed amount by number of unique patients, and/or billed amount standard deviation by mean.

The reports screen 1100 may further include a breakdown of costs 1110 by one or more comorbidity scores. To that end, the system may employ one or more pipelines to determine a comorbidity score for each patient. In one embodiment, the comorbidity score may be calculated via a pipeline associated with a node graph and context information that, when taken together, model the Charlson Comorbidity Index (“CCI”). The CCI is described in detail in Charlson, Mary E., et al. “A New Method of Classifying Prognostic Comorbidity in Longitudinal Studies: Development and Validation,” Journal of Chronic Diseases, 5:40 (1986), pp. 373-383, incorporated by reference herein in its entirety.

Upon calculating a comorbidity score, the system may determine and display one or more of: the total number of patients by comorbidity score 1111, the total costs by comorbidity scores 1112, the monthly costs by comorbidity scores, and the total cost per patient by comorbidity score 1114. Although not shown, the system may also determine and display a monthly cost per patient by comorbidity and/or a total cost over a given time period by comorbidity 1113 (e.g., for each Elixhauser comorbidity).

In one embodiment, the reports screen 1100 may include various user interface elements showing how costs and/or payments are spread among patients (i.e., what portion of costs are tied to what percentage of patients) 1115. Such interface elements may include charts and tables showing a percentage of total amount billed per percentage of patient population over one or more time periods 1116; charts and tables showing a top percentage of billed patients over one or more time periods 1117; charts and tables showing a percentage of total payments received per percentage of patient population over one or more time periods 1118; charts and tables showing a top percentage of paid clients over one or more time periods 1119; a table showing the costliest patients over a given time period 1120, including total amount billed 1122 and total payments received 1123 for each patient; and/or one or more patient-specific charts 1121 showing the date and amount of each billed amount and received payment.

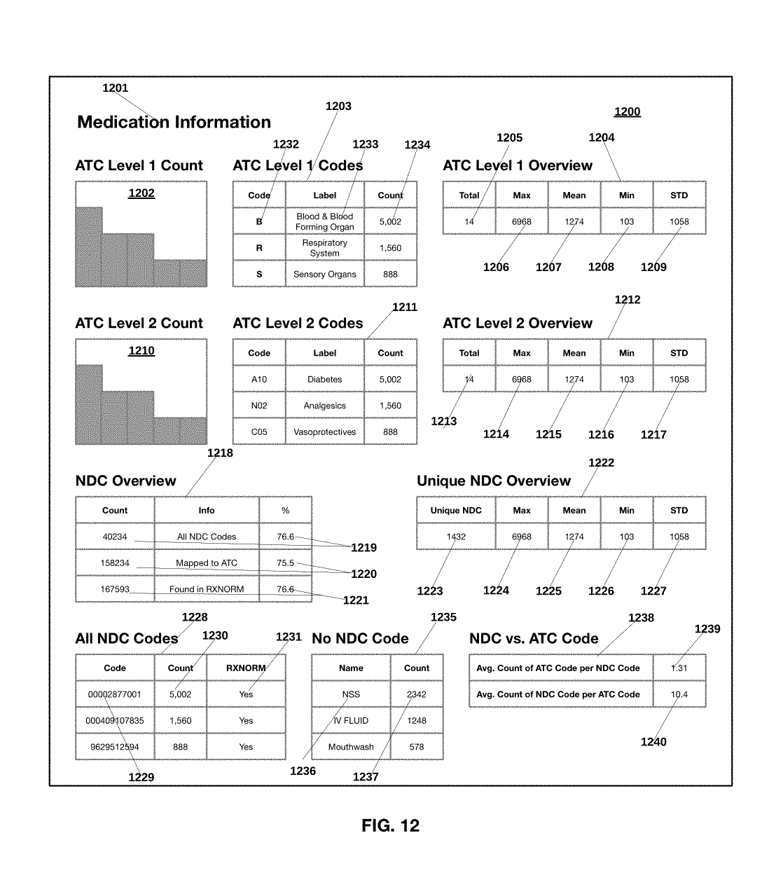

Referring to FIG. 12, an exemplary reports screen 1200 is illustrated, wherein the screen displays relevant medications information 1201 contained in input data, as determined via one or more pipelines. As shown, charts and tables may be provided to display each of the Anatomical Therapeutic Chemical (“ATC”) drug classification system codes 1203 and 1211 found in the input data. ATC classifications are available online from the World Health Organization (“WHO”), and are updated and published once a year by the WHO Collaborating Centre for Drug Statistics Methodology. See whocc.no/atc_ddd_index/.

In certain embodiments, separate tables/charts may be generated and displayed for each of the five ATC levels, including Level 1 (Anatomical Main Group) (1202-1204), Level 2 (Therapeutic Main Group) (1210-1212), Level 3 (Therapeutic/Pharmacological Subgroup), Level 4 (Chemical/Therapeutic/Pharmacological Subgroup) and/or Level 5 (Chemical Substance). As an example, a table and/or chart 1203 may show each of the ATC Level 1 codes 1232 found in the input data along with corresponding labels 1233 and a total count 1234. Similar interface elements may be generated and displayed for ATC Level 2 (1210-1212), Level 3, Level 4 and/or Level 5 codes.

As another example, an ATC Level 1 codes overview table 1204 may be provided to show one or more of: the total number of ATC Level 1 codes 1205, the minimum count of any ATC Level 1 code across all ATC Level 1 codes 1208, the maximum count of any ATC Level 1 code across all ATC Level 1 codes 1206, the mean count of ATC Level 1 codes across all ATC Level 1 codes 1207, the standard deviation of ATC Level 1 codes across all ATC Level 1 codes 1209. Similar overview tables may be provided for ATC Level 2 (1213-1217), Level 3 and/or Level 4 codes.

In one embodiment, the reports screen 1200 may include user interface elements to display information relating to National Drug Code (“NDC”) directory codes (1218-1222) identified in the input data (e.g., via one or more pipelines). The NDC directory is maintained by the U.S. Food & Drug Administration (“FDA”) according to Section 510 of the Federal Food, Drug, and Cosmetic Act (21 U.S.C. § 360) and is available at the following FDA website: fda.gov/Drugs/InformationOnDrugs/ucm142438.htm.

As shown, the system may display an overview table 1218 showing the total number of NDC codes found 1219, the number (or percentage) of found NDC codes that may be mapped by a pipeline to an ATC code 1220, and the number (or percentage) of found NDC codes that may be found in RxNORM 1221 (i.e., a normalized naming system for generic and branded drugs maintained by the U.S. National Library of Medicine). The system may further display a unique NDC overview table 1222, which includes the number of unique NDC codes found 1223, and any of the maximum 1224, minimum 1225, mean 1226, and/or standard deviation 1227 across each of the unique NDC codes.

The reports screen 1200 may further display a table 1228 of found NDC codes 1229, which includes a total count of each code 1230 and whether each code may be found in RxNORM 1231. The system may also show any prescribed medications found in the input data for which no NDC code is present 1235, including the name 1236 and total count 1237 for each medication. Finally, in certain embodiments, the system may include a table 1238 showing the average count of ATC codes per NDC codes 1239 and/or the average count of NDC codes per ATC code 1240.

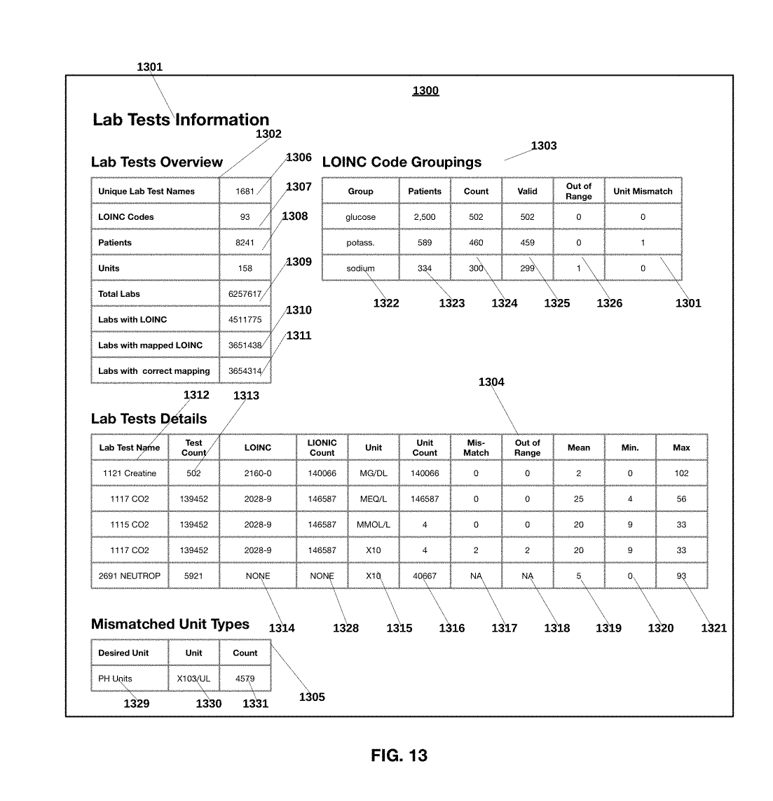

Referring to FIG. 13, an exemplary reports screen 1300 displaying lab tests information 1301 is illustrated. In one embodiment, the system may employ one or more pipelines to identify each of the lab test codes found in the input data and to map such codes to a corresponding Logical Observation Identifiers Names and Codes (“LOINC”) code. A database of LOINC codes is maintained by Regenstrief Institute, Inc. and may be accessed at loinc.org/downloads.

Upon mapping lab tests to LOINC codes, the system may display various user interface elements, such as a lab tests overview table 1302, a LOINC code groupings table 1303, a lab tests details table 1304 and a mismatched unit types table 1305. As shown, a lab tests overview table 1302 may be provided to show the number of unique lab test names found 1306, the total number of unique LOINC codes to which the lab tests are mapped 1307, the total number of patients associated with at least one lab test 1308, the total number of lab tests found 1309, the total number of lab tests that may be mapped to a LOINC code 1310 and/or the number of lab tests with correct LOINC mappings 1311.

The reports screen may also display a lab tests details table 1304, which includes each of the lab tests found in the input data. For each lab test in the table, corresponding information may be shown, such as: lab test name 1312, the total count of the lab test 1313, a corresponding LOINC code 1314, the LOINC count 1328, the expected unit 1315, the total number of times the expected unit is found in the input data 1316, an indication of how many occurrences of the lab test include a unit that is different than the expected unit 1317, an indication of how many occurrences of the lab test include a value that is outside of an expected range of values 1318 and/or the mean 1319/minimum 1320/maximum 1321/standard deviation value of the lab test across all occurrences.

In one embodiment, the system may provide a table of LOINC groupings 1303, where each grouping aggregates a number of related LOINC codes. Such table may include a list of LOINC groupings 1322 along with corresponding information, such as: the total number of unique patients associated with the grouping 1323 (i.e., with at least one of the LOINC codes associated with the grouping), the total number of lab tests mapped to each grouping 1324, the total number of valid lab tests associated with each grouping 1325, the total number of lab tests associated with the grouping that include at least one value that is out of an expected range 1326 (e.g., based on the individual LOINC codes), and the total number of lab tests associated with the group that include a value having a unit that is different than an expected unit (e.g., based on the LOINC code) 1327.

Finally, the reports screen 1300 may also include a mismatched unit types table 1305. As shown, this table may display any lab tests found 1331 in the input data that include a unit type 1330 that is different than an expected unit type 1329 (e.g., based on a mapped LOINC code).

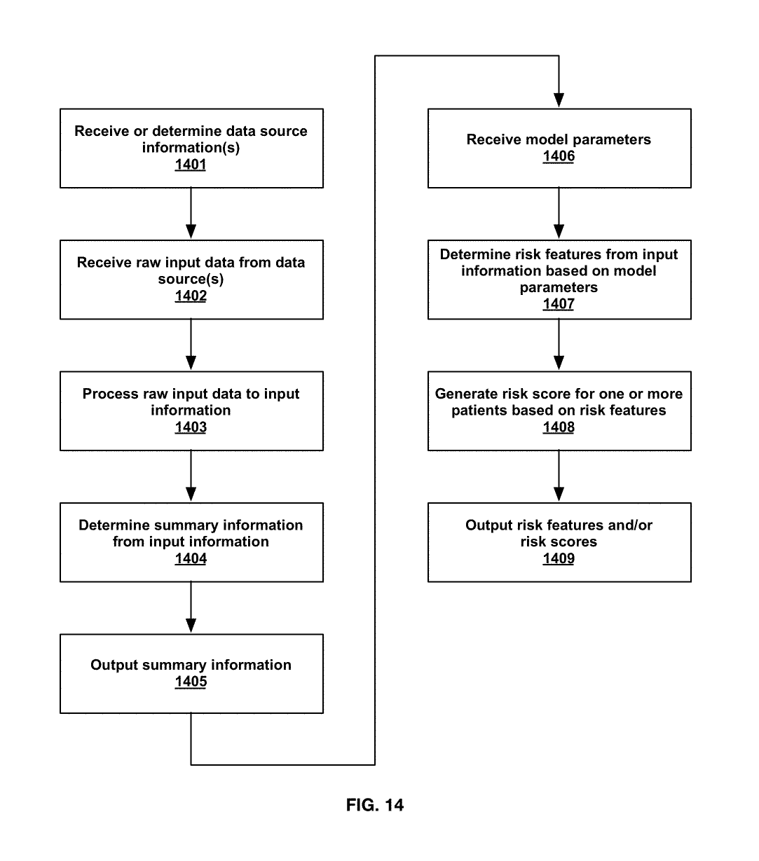

Referring to FIG. 14, an exemplary method is illustrated.

At step 1401 data source information is received by the system. Exemplary data source information may include a location where raw input data is stored and/or a type of data stored in the data source.

At step 1402, the system receives and stores raw input data from the one or more data sources and at step 1403 the system processes the raw input data into input information that may be stored. As discussed in detail above, such processing may employ one or more pipelines associated with any number of nodes that validate, cleanse and/or normalize the raw input data. Exemplary processing steps may include converting various codes to standard codes, encoding categorical variables, normalizing continuous variables, log scaling count variables, bucketing, binning, determining values (e.g., maximums, minimums, means, medians, modes, etc.) and/or combining data as necessary to create data tables having a standardized format or schema.

As discussed in detail above in reference to FIGS. 10-13, the system may employ pipelines to determine summary information from the stored input information (1404) and may output some or all of the summary information as a report 1405.

Embodiments of the described platforms may also employ various pipelines to help organizations understand risk factors that lead to adverse events and to determine which patients are at an increased risk of experiencing adverse events in the future.

Accordingly, the system may receive any number of modeling parameters 1406 that may be used to create, train and validate a predictive engine. Such parameters may include target events or outcomes for which predictions are to be made, a prediction period (e.g., a period beginning on a certain date during which the target event/outcome may occur), and/or an observation period (e.g., a period before the prediction period from which data may be used to train and validate the model).

Generally, the system may employ machine learning algorithms (e.g., random forest classifier, logistic regression, DNN classifier, etc.) to determine important risk factors for various adverse event/outcomes 1407 (e.g., features and meta-features of the input data), and/or to predict the likelihood that particular patients will experience such adverse events (e.g., via a risk score) 1408. The platform may then output risk information 1409, such as risk factors and patient risk scores, in the form of downloadable reports and/or online dashboards.

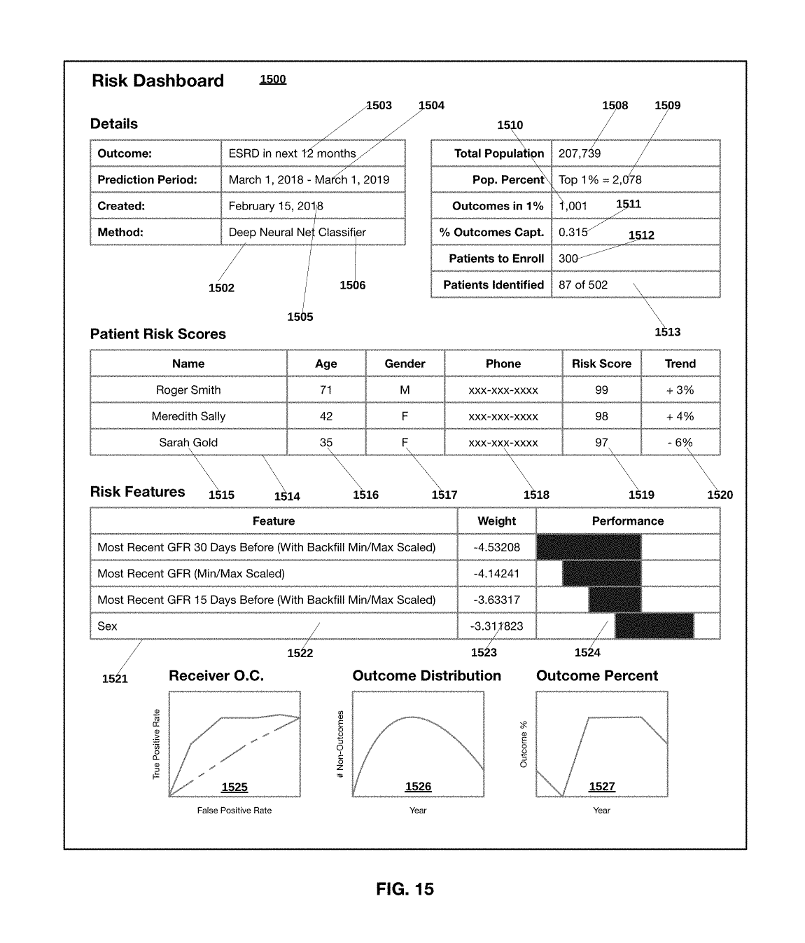

Referring to FIG. 15, an exemplary risk report screen 1500 is illustrated. The risk report screen may display various risk information determined by the predictive engine from input data and/or information relating to predictive engine performance. As shown, this screen may display a details table 1502, a patient risk scores table 1514, and a risk features table 1521.

In one embodiment, the report may include information about the predictive engine itself and the input data analyzed by the engine. For example, the report displays: the target outcome/event for which predictions were made 1503 (e.g., End-Stage Renal Disease (“ESRD”)), the corresponding prediction period 1504, a date the prediction was made 1505, and the machine learning algorithm 1506 that was employed to make the prediction. The report may further display the total number of patients found in the input data 1508, the number of patients in the top 1% 1509, the total number of patients in the top 1% who are predicted to experience the outcome 1510, the percent of outcomes captured 1511, the number of patients to enroll 1512 and the number of identified patients 1513.

The risk reports screen 1500 may also display a patient risk scores table 1514, which displays the patients who are the greatest risk of experiencing the outcome (i.e., patients with the highest risk score), along with corresponding patient information. As shown, the table may display the following information for each patient: name 1515, age 1516, gender 1517, contact information 1518, risk score 1519, and/or the trend over a predetermined period of time of the patient's risk score 1520.

The reports screen may also display a risk features table 1521, which shows each of the features 1522 employed by the predictive engine to make predictions. In one embodiment, the table may include information relating to the performance of each feature 1524 and/or the weight 1523 applied to each feature by the predictive engine to make predictions.

Finally, the reports screen may also display various interface elements providing information about the input data. For example, the screen may display a receiver operating characteristics (“ROC”) graph 1525 showing the ROC curve and corresponding area; an outcome distribution graph 1526 showing the total number of non-outcomes per year; and an outcome percent graph 1527 depicting the percentage of adverse outcomes per year.

Various embodiments are described in this specification, with reference to the detailed discussed above, the accompanying drawings, and the claims. Numerous specific details are described to provide a thorough understanding of various embodiments. However, in certain instances, well-known or conventional details are not described in order to provide a concise discussion. The figures are not necessarily to scale, and some features may be exaggerated or minimized to show details of particular components. Therefore, specific structural and functional details disclosed herein are not to be interpreted as limiting, but merely as a basis for the claims and as a representative basis for teaching one skilled in the art to variously employ the embodiments.

The embodiments described and claimed herein and drawings are illustrative and are not to be construed as limiting the embodiments. The subject matter of this specification is not to be limited in scope by the specific examples, as these examples are intended as illustrations of several aspects of the embodiments. Any equivalent examples are intended to be within the scope of the specification. Indeed, various modifications of the disclosed embodiments in addition to those shown and described herein will become apparent to those skilled in the art, and such modifications are also intended to fall within the scope of the appended claims.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

All references including patents, patent applications and publications cited herein are incorporated herein by reference in their entirety and for all purposes to the same extent as if each individual publication or patent or patent application was specifically and individually indicated to be incorporated by reference in its entirety for all purposes.

What is claimed is:

1. A computer-implemented method comprising:
receiving, by a computer, raw input data associated with a first format;
storing, by the computer, the raw input data in a first memory;
storing, by the computer, a plurality of data nodes, each of the data nodes adapted to:
receive an input; and
manipulate the input according to an associated functionality to generate an output;
storing, by a computer, a context object associated with a pipeline, the context object including context information comprising:
one or more input nodes selected from the plurality of data nodes, the input nodes adapted to:
receive the raw input data stored in the first memory; and
manipulate the raw input data according to the functionality associated with each of the input nodes to generate standardized data associated with a standardized format that is different than the first format;
one or more processing nodes selected from the plurality of data nodes, the processing nodes adapted to:
receive the standardized data;
manipulate the standardized data according to the functionality associated with each of the processing nodes to generate output data; and
relationship information corresponding to:
how each of the input nodes is connected to one or more other input nodes;
how at least one of the input nodes is connected to at least one of the processing nodes; and
how each of the processing nodes is connected to one or more other processing nodes;
receiving, by the computer, a data processing request associated with the pipeline and the raw input data; and
upon receiving the request:
creating, by the computer, a node graph based on the context information, the node graph comprising the input nodes and the processing nodes,
wherein at least one of the input nodes is linked to the first memory such that the raw input data is received therefrom, and
wherein at least one of the processing nodes is linked to at least one of the input nodes such that the standardized data is received therefrom;
processing, by the computer, the raw input data to the output data via the node graph; and
storing, by the computer, the output data.

2. A computer-implemented method according to claim 1, wherein the functionality associated with each of the plurality of data nodes is selected from the group consisting of: joining, filtering, aggregating, caching, counting, renaming, searching, sorting, calculating a value, determining a maximum, determining a minimum, determining a mean, and/or determining a standard deviation.

3. A computer-implemented method according to claim 1, wherein the node graph comprises a direct acyclic graph (“DAG”).

4. A computer-implemented method according to claim 1, further comprising generating a report comprising the output data.

5. A computer-implemented method according to claim 4, wherein:
the report is associated with a Uniform Resource Locator (“URL”); and
the report is displayed to a user via the URL.

6. A computer-implemented method according to claim 4, wherein the report comprises a downloadable digital file.

7. A computer-implemented method according to claim 1, wherein the raw input data comprises one or more of: patient demographics information, insurance claims information, diagnoses information, medical procedures information, lab test results, medications information, genomics information, and financial information.

8. A computer-implemented method according to claim 1, wherein said processing comprises:
scheduling, by the computer, a plurality of tasks, based on the node graph;
associating, by the computer, each of the plurality of tasks with scheduling information; and
assigning, by the computer, each of the plurality of tasks to a computing resource such that each task is executed by the respective computing resource according to the associated scheduling information.

9. A computer-implemented method according to claim 1, wherein:
the context information further comprises one or more second input nodes selected from the plurality of data nodes, the second input nodes adapted to:
receive second raw input data associated with a second format that is different than both the first format and the standardized format; and
manipulate the second raw input data according to the functionality associated with each of the second input nodes to generate the standardized data; and
the relationship information further corresponds to how each of the second input nodes is connected to one or more other second input nodes.

10. A computer-implemented method according to claim 9 further comprising:
receiving, by the computer, the second raw input data;
storing, by the computer, the second raw input data in a second memory;
receiving, by the computer, a second data processing request associated with the pipeline and the second raw input data;
upon receiving the second request:
creating, by the computer, a second node graph based on the context information, the second node graph comprising the second input nodes and the processing nodes,
wherein at least one of the second input nodes is linked to the second memory such that the second raw input data is received therefrom, and
wherein at least one of the processing nodes is linked to at least one of the second input nodes such that the standardized data is received therefrom; and
processing, by the computer, the second raw input data to the output data via the second node graph.

11. A computer-implemented method according to claim 1, wherein the context information further comprises caching information.

12. A computer-implemented method according to claim 11, wherein:
the caching information corresponds to an instruction to store the standardized data output by the input nodes; and
said processing the raw input data to the output data via the node graph comprises storing the standardized data, based on the caching information.

13. A computer-implemented method according to claim 1, further comprising:
receiving, by the computer, a second request to filter the output data;
traversing the node graph backwards from an end node to determine a selected node that can fulfill the second request;
upon determining the selected node, updating the node graph to include a filtering node that depends from the selected node.

14. A computer-implemented method according to claim 1, further comprising:
searching the standardized data;
determining that the standardized data contains first patient information corresponding to a first patient;
creating a first record corresponding to the first patient, the first record comprising the first patient information;
calculating a first risk score for the first patient, based on the first record and a plurality of risk factors, the risk score relating to a predicted probability that the patient will experience an adverse event within a predetermined amount of time in the future; and
outputting the first risk score.

15. A computer-implemented method according to claim 14, further comprising:
determining that the standardized data contains second patient information corresponding to the first patient;
updating the first record to include the second patient information;
calculating an updated first risk score for the first patient, based on the updated first record and the plurality of risk factors; and
outputting the updated first risk score.

16. A computer-implemented method according to claim 14, further comprising:
determining that the standardized data contains second patient information corresponding to a second patient;
creating a second record corresponding to the second patient, the second record comprising the second patient information;
calculating a second risk score for the second patient, based on the second record and the plurality of risk factors; and
outputting the second risk score.

17. A computer-implemented method according to claim 16, wherein said outputting the first and second risk scores comprises generating a report that includes the first and second risk scores.

18. A computer-implemented method according to claim 17, wherein:
said calculating the first risk score comprises:
calculating a first value for each of the plurality of risk factors, based on the first record;
applying a risk-factor-specific weight to each of the calculated first values; and
adding the weighted first values together to thereby calculate the first risk score; and
said calculating the second risk score comprises:
calculating a second value for each of the plurality of risk factors, based on the second record;
applying the risk-factor-specific weight to each of the calculated second values; and
adding the weighted second values together to thereby calculate the second risk score.

19. A system comprising one or more processing units, and one or more processing modules, wherein the system is configured by the one or more processing modules to:
receive raw input data associated with a first format;
store the raw input data in a first memory;
store a plurality of data nodes, each of the data nodes adapted to:
receive an input; and
manipulate the input according to an associated functionality to generate an output;
store a context object associated with a pipeline, the context object including context information comprising:
one or more input nodes selected from the plurality of data nodes, the input nodes adapted to:
receive the raw input data stored in the first memory; and
manipulate the raw input data according to the functionality associated with each of the input nodes to generate standardized data associated with a standardized format that is different than the first format;
one or more processing nodes selected from the plurality of data nodes, the processing nodes adapted to:
receive the standardized data;
manipulate the standardized data according to the functionality associated with each of the processing nodes to generate output data; and
relationship information corresponding to:
how each of the input nodes is connected to one or more other input nodes;
how at least one of the input nodes is connected to at least one of the processing nodes; and
how each of the processing nodes is connected to one or more other processing nodes;
receive a data processing request associated with the pipeline and the raw input data; and
upon receiving the request:
create a node graph based on the context information, the node graph comprising the input nodes and the processing nodes,
wherein at least one of the input nodes is linked to the first memory such that the raw input data is received therefrom, and
wherein at least one of the processing nodes is linked to at least one of the input nodes such that the standardized data is received therefrom;
process the raw input data to the output data via the node graph; and
store the output data.

20. A system according to claim 19, wherein:
the context information further comprises one or more second input nodes selected from the plurality of data nodes, the second input nodes adapted to:
receive second raw input data associated with a second format that is different than both the first format and the standardized format; and
manipulate the second raw input data according to the functionality associated with each of the second input nodes to generate the standardized data;
the relationship information further corresponds to how each of the second input nodes is connected to one or more other second input nodes; and
the system is further configured by the one or more processing modules to:
receive the second raw input data;
store the second raw input data in a second memory;
receive a second data processing request associated with the pipeline and the second raw input data;
upon receiving the second request:
create a second node graph based on the context information, the second node graph comprising the second input nodes and the processing nodes,
wherein at least one of the second input nodes is linked to the second memory such that the second raw input data is received therefrom, and
wherein at least one of the processing nodes is linked to at least one of the second input nodes such that the standardized data is received therefrom; and
process the second raw input data to the output data via the second node graph.
